(19) United States
(12) Patent Application Publication
(10) Pub. No.: US 2023/0158062 A1
(43) Pub. Date: May 25, 2023

(54) FORMULATIONS FOR TREATING CONSTIPATION

(71) Applicant: ColonaryConcepts LLC, Palm Beach Gardens, FL (US)

(72) Inventors: Corey A. SIEGEL, Palm Beach Gardens, FL (US); Michael K. ALLIO, Palm Beach Gardens, FL (US)

(21) Appl. No.: 16/084,173

(22) PCT Filed: Mar. 28, 2017
(86) PCT No.: PCT/US2017/024606

§ 371 Date: Sep. 11, 2018

(51) Int. Cl. A61K 31/765; A23L 33/25; A61K 9/00

(52) U.S. Cl. CPC A61K 31/765; A23L 33/25; A61K 9/0056

(57) ABSTRACT

Described herein are compositions and methods for treating constipation in an individual. In some embodiments, the composition and method for treating constipation comprises providing an individual with a therapeutic that comprises a food item and a laxative.

### CROSS-REFERENCE

This application is filed pursuant to 35 U.S.C. § 371 as a United States National Phase Application of International Application No. PCT/US2017/024606 entitled “FORMULATIONS FOR TREATING CONSTIPATION” filed Mar. 28, 2017, which claims the benefit of U.S. Provisional Application No. 62/314,883, filed Mar. 29, 2016, which are incorporated herein by reference in their entirety.

### BACKGROUND

Constipation is a widespread condition worldwide. Constipation is associated with flatus and intestinal swelling, which causes discomfort and abdominal pain to sufferers.

There are numerous causes of constipation including medical conditions and iatrogenic constipation resulting from certain prescribed medications.

### SUMMARY

Constipation affects millions of people yearly worldwide. The condition is attributed to numerous causes, including certain medical conditions and physiological conditions such as Irritable Bowel Syndrome, GI motility disorders, pregnancy, sedentary lifestyle, and diet. In addition numerous medications are associated with causing constipation as a side-effect.

Traditional laxative based therapies for constipation do not provide a palatable, convenient, appealing, or easily portable method for treating constipation. As such, traditional laxative based therapies do not provide a mechanism for encouraging individual compliance with the treatment.

Described herein are compositions and methods that overcome short comings in traditional therapies by providing a delivery vehicle for one or more laxatives that provides optimal delivery of the one or more laxatives to the colon in order to optimally treat constipation, and further providing a satisfying experience for the individual ingesting the composition thus promoting compliance with the treatment. In some embodiments, the delivery vehicle is a food based delivery vehicle.

Described herein are compositions and methods for promoting gastrointestinal motility. Chewing is known to promote gastrointestinal motility and as such a food based delivery vehicle of a constipation therapeutic as described herein, in solid form, promotes chewing when ingested and as such promotes gastrointestinal motility.

Described herein are compositions and methods for treating constipation in an individual. In some embodiments, the composition and method for treating constipation comprises providing an individual with a therapeutic that comprises a food item.

Described herein is a method for treating constipation in an individual comprising: providing the individual with a food item comprising one or more food ingredients and one or more laxatives, wherein ingestion of the food item by the individual produces a bowel movement in the individual and thus relieves constipation in the individual. In some embodiments, the food item comprises a shelf-stable packaged solid food item. In some embodiments, the one or more laxatives wherein the one or more laxatives comprises any of dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG), vitamin C, dioctyl sulfosuccinate (Docusate), bisacodyl (Dulcolax), castor oil, and sorbitol. In some embodiments, the PEG comprises PEG 3350 in a quantity of 17 grams or less. In some embodiments, the one or more food ingredients are mixed together with the one or more laxatives. In some embodiments, the one or more laxatives comprises a coating of the food item. In some embodiments, the bowel movement in the individual is produced within 12 hours of ingestion of the one or more laxatives. In some embodiments, the food item comprises a food bar. In some embodiments, the food item comprises a shake or similar beverage. In some embodiments, the individual uses a medication, a side effect of which is constipation. In some embodiments, the medication comprises an opioid. In some embodiments, the opioid comprises one or more of morphine, codeine, oxycodone, and dilaudid. In some embodiments, the medication comprises an antidepressant. In some embodiments, the antidepressant comprises one or more of amitriptyline and imipramine. In some embodiments, the medication comprises an anticonvulsant. In some embodiments, the anticonvulsant comprises one or more of phenytoin and carbamazepine. In some embodiments, the medication comprises an iron supplement. In some embodiments, the medication comprises a calcium channel blocker. In some embodiments, the calcium channel blocker comprises one or more of diltiazem and nifedipine. In some embodiments, the constipation has lasted for a duration of 24 hours or more. In some embodiments, the food item comprises a meal.

Described herein is a composition comprising, one or more laxatives; one or more food ingredients; wherein the laxative is combined with the one or more food ingredients, and wherein the one or more food ingredients are either of a quantity or of a type such that when the composition is ingested by an individual, the one or more food ingredients will be digested to an extent that the one or more laxatives will be released within a colon of the individual. In some embodiments, the one or more laxatives comprises polyethylene glycol (PEG) 3350. In some embodiments, the PEG 3350 comprises a quantity of 17 grams or less. In some embodiments, the one or more food ingredients are mixed together with the one or more laxatives. In some embodiments, the one or more food ingredients comprise a coating of the composition. In some embodiments, a bowel movement in the individual is produced within 12 hours of ingestion of the composition. In some embodiments, the composition comprises a ready to eat food bar. In some embodiments, the composition comprises a ready to drink shake. In some embodiments, the composition comprises an edible powder configured to be mixed with a liquid. In some embodiments, the composition comprises a meal. In some embodiments, the one or more laxatives are 50% or greater by weight of a total weight of the composition.

### DETAILED DESCRIPTION

Described herein are compositions and methods for treating constipation in an individual.

### Compositions for Treating Constipation

Described herein are compositions for treating constipation in an individual comprising a food item.

In some embodiments, the food item comprises any solid or liquid food item. A solid or liquid food item may comprise a discrete food item or a meal. Non-limiting examples of solid food items include food bars, baked goods, meat products, fruit and fruit products, vegetables and plant based products, candies, gums, nuts and nut products. Non-limiting examples of liquid food items include shakes, sodas, milks, coffees, teas, and flavored water based drinks.

In some embodiments, the food item comprises food ingredients that may comprise any edible food ingredients used to make a food item. In some embodiments, the food item comprises one or more ingredients that comply with the standards of the National Formulary of the U.S. Pharmacopeial Convention (USP-NF). Non-limiting examples of ingredients include sugars and natural sweeteners, artificial sweeteners, grain based flour, non-grain based flour, fruit and fruit products, vegetables and vegetable products, dairy products including milk, cream, cheese, and butter, edible oils, chocolate, and nuts.

A food item as described herein comprises one or more food ingredients combined with one or more laxatives. Non-limiting examples of laxatives suitable for combination with the one or more food ingredients include dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG) (including, for example, PEG 3350, PEG 4000, PEG 6000, and PEG 8000), vitamin C, dioctyl sulfosuccinate (Docusate), bisacodyl (Dulcolax), castor oil, and sorbitol.

In some embodiments, one or more food ingredients and one or more laxatives are combined so that the laxative is incorporated inside of the food item. For example, in some embodiments, the food item comprises a food bar and one or more laxatives are incorporated within the food bar. In some embodiments, one or more food ingredients are mixed together with one or more laxatives to form the food item. In some embodiments, the one or more food ingredients and the one or more laxatives form a homogenous mixture. In some embodiments, the one or more food ingredients and one or more laxatives do not comprise a homogenous mixture.

In some embodiments, one or more laxatives comprise one or more agglomerated collections of laxative that are at least partially surrounded by one or more food ingredients. For example, in some embodiments, a single agglomerated quantity of PEG 3350 is surrounded by one or more food ingredients of a baked good such as a brownie so that the agglomerated quantity of PEG 3350 is incorporated within the brownie which comprises the food item. In some embodiments, one or more laxatives comprises a coating of an agglomerated quantity of food ingredients. For example, in some embodiments, a food item comprises a bar with a coating comprising of one or more laxatives. In some embodiments, a coating comprises an icing, frosting, or fondant.

In some embodiments, a laxative combined with one or more food ingredients comprises PEG 3350. In some embodiments, the quantity of PEG 3350 combined with one or more food ingredients comprises 130 grams of PEG 3350. In some embodiments, the quantity of PEG 3350 combined with one or more food ingredients comprises 120 grams of PEG 3350. In some embodiments, the quantity of PEG 3350 combined with one or more food ingredients comprises 110 grams of PEG 3350. In some embodiments, the quantity of PEG 3350 combined with one or more food ingredients comprises 100 grams of PEG 3350. In some embodiments, the quantity of PEG 3350 combined with one or more food ingredients comprises 90 grams of PEG 3350. In some embodiments, the quantity of PEG 3350 combined with one or more food ingredients comprises 80 grams of PEG 3350. In some embodiments, the quantity of PEG 3350 combined with one or more food ingredients comprises 70 grams of PEG 3350. In some embodiments, the quantity of PEG 3350 combined with one or more food ingredients comprises 60 grams of PEG 3350. In some embodiments, the quantity of PEG 3350 combined with one or more food ingredients comprises 50 grams of PEG 3350. In some embodiments, the quantity of PEG 3350 combined with one or more food ingredients comprises 40 grams of PEG 3350. In some embodiments, the quantity of PEG 3350 combined with one or more food ingredients comprises 30 grams of PEG 3350. In some embodiments, the quantity of PEG 3350 combined with one or more food ingredients comprises 20 grams of PEG 3350. In some embodiments, the quantity of PEG 3350 combined with one or more food ingredients comprises 19 grams of PEG 3350. In some embodiments, the quantity of PEG 3350 combined with one or more food ingredients comprises 18 grams of PEG 3350. In some embodiments, the quantity of PEG 3350 combined with one or more food ingredients comprises 17 grams of PEG 3350. In some embodiments, the quantity of PEG 3350 combined with one or more food ingredients comprises 16 grams of PEG 3350. In some embodiments, the quantity of PEG 3350 combined with one or more food ingredients comprises 15 grams of PEG 3350. In some embodiments, the quantity of PEG 3350 combined with one or more food ingredients comprises 14 grams of PEG 3350. In some embodiments, the quantity of PEG 3350 combined with one or more food ingredients comprises 13 grams of PEG 3350. In some embodiments, the quantity of PEG 3350 combined with one or more food ingredients comprises 12 grams of PEG 3350. In some embodiments, the quantity of PEG 3350 combined with one or more food ingredients comprises 11 grams of PEG 3350. In some embodiments, the quantity of PEG 3350 combined with one or more food ingredients comprises 10 grams of PEG 3350. In some embodiments, the quantity of PEG 3350 combined with one or more food ingredients comprises 9 grams of PEG 3350. In some embodiments, the quantity of PEG 3350 combined with one or more food ingredients comprises 8 grams of PEG 3350. In some embodiments, the quantity of PEG 3350 combined with one or more food ingredients comprises 7 grams of PEG 3350. In some embodiments, the quantity of PEG 3350 combined with one or more food ingredients comprises 6 grams of PEG 3350. In some embodiments, the quantity of PEG 3350 combined with one or more food ingredients comprises 5 grams of PEG 3350. In some embodiments, the quantity of PEG 3350 combined with one or more food ingredients comprises 9 grams of PEG 3350. In some embodiments, the quantity of PEG 3350 combined with one or more food ingredients comprises 8 grams of PEG 3350. In some embodiments, the quantity of PEG 3350 combined with one or more food ingredients comprises 7 grams of PEG 3350. In some embodiments, the quantity of PEG 3350 combined with one or more food ingredients comprises 6 grams of PEG 3350. In some embodiments, the quantity of PEG 3350 combined with one or more food ingredients comprises 5 grams of PEG 3350. In some embodiments, the quantity of PEG 3350 combined with one or more food ingredients comprises 4 grams of PEG 3350. In some embodiments, the quantity of PEG 3350 combined with one or more food ingredients comprises 3 grams of PEG 3350. In some embodiments, the quantity of PEG 3350 combined with one or more food ingredients comprises 2 grams of PEG 3350. In some embodiments, the quantity of PEG 3350 combined with one or more food ingredients comprises 1 grams of PEG 3350.

In some embodiments, a composition for treating composition comprises 40% or greater laxative by weight. In some embodiments, a composition for treating composition comprises 45% or greater laxative by weight. In some embodiments, a composition for treating composition comprises 50% or greater laxative by weight. In some embodiments, a composition for treating composition comprises 55% or greater laxative by weight. In some embodiments, a composition for treating composition comprises 60% or greater laxative by weight. In some embodiments, a composition for treating composition comprises 65% or greater laxative by weight. In some embodiments, a composition for treating composition comprises 70% or greater laxative by weight. In some embodiments, a composition for treating composition comprises 75% or greater laxative by weight. In some embodiments, a composition for treating composition comprises 80% or greater laxative by weight. In some embodiments, a composition for treating composition comprises 85% or greater laxative by weight. In some embodiments, a composition for treating composition comprises 90% or greater laxative by weight. In some embodiments, a composition for treating composition comprises 95% or greater laxative by weight.

In some embodiments, the location of delivery of the therapeutic agent for treating constipation within the gastrointestinal tract is related to the rate of digestion of the composition in which the therapeutic is incorporated. For example, a composition comprising a thin edible layer takes relatively less time to digest and is digested in the proximal part of the intestinal tract releasing the therapeutic agent for treating constipation in the proximal part of the intestinal tract. For example, a composition comprising a thick edible layer takes relatively more time to digest and is digested in the distal portion of the digestive tract. Thus, by, for example, controlling the thickness of the composition, a point of release of the therapeutic agent for treating constipation within the gastrointestinal tract is controlled. Similarly, in some embodiments, the composition comprises ingredients that are digested in the proximal portion of the gastrointestinal tract, and in some embodiments, the composition comprises ingredients that are digested in the distal portion of the gastrointestinal tract.

Table 1 below shows a formulation for an exemplary composition for treating constipation comprising a lemon bar.

TABLE 1

| | | Amount per |
|---|---|---|
| Lemon Bar | | Unit (grams) |
| Ingredient | % by weight | 24.4 |
| Polyethylene Glycol 3350 | 69.8% | 17.023 |
| Invert Syrup | 17.0% | 4.142 |
| Cocoa Butter | 12.4% | 3.018 |
| Flavor, Milky Vanilla N&A #2713, | 0.7% | 0.171 |
| David Michael | | |
| Citric Acid | 0.1% | 0.029 |
| Lemon Oil | 0.1% | 0.016 |
| | 100.0% | 24.400 |

As shown in table 1, a lemon bar comprises 69.8% by weight PEG 3350 with a total amount of PEG of 17.023 grams. One or more food ingredients include invert syrup, cocoa butter, vanilla, citric acid, and lemon oil. In some embodiments, one or more ingredients are USP-NF grade

Table 2 below shows a formulation for an exemplary composition for treating constipation comprising a white chocolate bar.

TABLE 2

| | | Amount per |
|---|---|---|
| White Chocolate Bar | | Unit (grams) |
| Ingredient | % by weight | 24.4 |
| Polyethylene Glycol 3350 | 69.767% | 17.023 |
| Invert Syrup | 16.772% | 4.092 |
| Cocoa Butter | 11.860% | 2.894 |
| Flavor, Milky Vanilla N&A | 0.700% | 0.171 |
| #2713, David Michael | | |
| Flavor, Chocolate N&A #50083, | 0.800% | 0.195 |
| David Michael | | |
| Flavor, Creamy Vanilla O.S. #50043, | 0.100% | 0.024 |
| David Michael | | |
| | 100.000% | 24.400 |

As shown in table 2, a white chocolate bar comprises 69.767% by weight PEG 3350 with a total amount of PEG of 17.023 grams. One or more food ingredients include invert syrup, cocoa butter, vanilla, and chocolate flavor. In some embodiments, one or more ingredients are USP-NF grade.

Table 3 below shows a formulation for an exemplary composition for treating constipation comprising a white chocolate bar.

TABLE 3

| | | Amount per |
|---|---|---|
| Coconut Bar | | Unit (grams) |
| Ingredient | % by weight | 24.4 |
| Polyethylene Glycol 3350 | 69.767% | 17.023 |
| Invert Syrup | 16.915% | 4.127 |
| Cocoa Butter | 12.267% | 2.993 |
| Flavor, Coconut #3728, David Michael | 0.800% | 0.195 |
| Flavor, Creamy Vanilla O.S. #50043, | 0.250% | 0.061 |
| David Michael | | |
| | 100.000% | 24.400 |

As shown in table 3, a coconut bar comprises 69.767% by weight PEG 3350 with a total amount of PEG of 17.023 grams. One or more food ingredients include invert syrup, cocoa butter, vanilla, and coconut flavor. In some embodiments, one or more ingredients are USP-NF grade.

Table 4 below shows a formulation for an exemplary composition for treating constipation comprising a strawberry banana shake.

TABLE 4

| | | Amount per |
|---|---|---|
| Strawberry Banana Beverage | | Unit (grams) |
| Ingredient | % by weight | 122.0 |
| Polyethylene Glycol, 3350 (powder) | 94.2623% | 115.00 |
| Sucrose, granulated USP | 5.2018% | 6.35 |
| Flavor. Strawberry Banana N&A #3620, | 0.4172% | 0.51 |
| David Michael | | |
| Citric acid, USP | 0.1188% | 0.14 |
| | 100.000% | 122.0 |

As shown in table 4, a strawberry banana shake comprises 94.2623% by weight PEG 3350 with a total amount of PEG of 115.0 grams. One or more food ingredients include USP-NF sucrose, strawberry banana flavor, and USP-NF citric acid.

Table 5 below shows a formulation for an exemplary composition for treating constipation comprising a vanilla beverage.

TABLE 5

| | | Amount per |
|---|---|---|
| Vanilla Beverage | | Unit (grams) |
| Ingredient | % by weight: | 120 |
| Polyethylene Glycol, 3350 (powder) | 95.833% | 115.00 |
| Sucrose, granulated USP | 2.500% | 3.00 |
| Flavor, Milky Vanilla N&A #2713, | 1.667% | 2.00 |
| David Michael | | |
| | 100.000% | 120.0 |

As shown in table 5, a vanilla beverage comprises 95.833% by weight PEG 3350 with a total amount of PEG of 115.0 grams. One or more food ingredients include USP-NF sucrose and vanilla flavor.

In some embodiments, the compositions described herein comprise portable ready to eat food items.

Non-limiting examples of flavors suitable for use in the compositions described herein include sweet flavors, savory flavors, spicy flavors, and sour flavors.

### Constipation Treatment

In some embodiments, the compositions and methods described herein are used to treat individual suffering from constipation. In some embodiments, the constipation treated by the compositions and methods described herein is caused by a disease process such as, for example, Irritable Bowel Syndrome. In some embodiments, the constipation treated by the compositions and methods described herein is caused by a disease process such as an anxiety disorder. In some embodiments, the constipation treated by the compositions and methods described herein is caused by aging. In some embodiments, the constipation treated by the compositions and methods described herein is caused by a wasting disease such as certain cancers. In some embodiments, the constipation treated by the compositions and methods described herein is caused by poor nutrition. In some embodiments, the constipation treated by the compositions and methods described herein is caused by a medication. In some embodiments, the constipation treated by the compositions and methods described herein is caused by an opioid. In some embodiments, the constipation treated by the compositions and methods described herein is caused by codeine. In some embodiments, the constipation treated by the compositions and methods described herein is caused by oxycodone. In some embodiments, the constipation treated by the compositions and methods described herein is caused by hydromorphone. In some embodiments, the constipation treated by the compositions and methods described herein is caused by buprenorphine. In some embodiments, the constipation treated by the compositions and methods described herein is caused by fentanyl. In some embodiments, the constipation treated by the compositions and methods described herein is caused by hydrocodone. In some embodiments, the constipation treated by the compositions and methods described herein is caused by meperidine. In some embodiments, the constipation treated by the compositions and methods described herein is caused by methadone. In some embodiments, the constipation treated by the compositions and methods described herein is caused by morphine sulfate. In some embodiments, the constipation treated by the compositions and methods described herein is caused by oxymorphone. In some embodiments, the constipation treated by the compositions and methods described herein is caused by tramadol. In some embodiments, the constipation treated by the compositions and methods described herein is caused by an antidepressant. In some embodiments, the constipation treated by the compositions and methods described herein is caused by amitriptyline. In some embodiments, the constipation treated by the compositions and methods described herein is caused by imipramine. In some embodiments, the constipation treated by the compositions and methods described herein is caused by doxepin. In some embodiments, the constipation treated by the compositions and methods described herein is caused by an antihypertensive. In some embodiments, the constipation treated by the compositions and methods described herein is caused by clonidine. In some embodiments, the constipation treated by the compositions and methods described herein is caused by a beta blocker. In some embodiments, the constipation treated by the compositions and methods described herein is caused by atenolol. In some embodiments, the constipation treated by the compositions and methods described herein is caused by an anti-Parkinson's agent. In some embodiments, the constipation treated by the compositions and methods described herein is caused by bromocriptine. In some embodiments, the constipation treated by the compositions and methods described herein is caused by a cholesterol lowering agent. In some embodiments, the constipation treated by the compositions and methods described herein is caused by cholestyramine. In some embodiments, the constipation treated by the compositions and methods described herein is caused by a medication for treatment of gastrointestinal ulcers. In some embodiments, the constipation treated by the compositions and methods described herein is caused by sucralfate. In some embodiments, the constipation treated by the compositions and methods described herein is caused by an anticonvulsant. In some embodiments, the constipation treated by the compositions and methods described herein is caused by phenytoin. In some embodiments, the constipation treated by the compositions and methods described herein is caused by carbamazepine. In some embodiments, the constipation treated by the compositions and methods described herein is caused by an iron supplement. In some embodiments, the constipation treated by the compositions and methods described herein is caused by a calcium channel blocker. In some embodiments, the constipation treated by the compositions and methods described herein is caused by diltiazem. In some embodiments, the constipation treated by the compositions and methods described herein is caused by nifedipine. In some embodiments, the constipation treated by the compositions and methods described herein is caused by an NSAID. In some embodiments, the constipation treated by the compositions and methods described herein is caused by ibuprofen. In some embodiments, the constipation treated by the compositions and methods described herein is caused by an anticholinergic. In some embodiments, the constipation treated by the compositions and methods described herein is caused by diphenhydramine. In some embodiments, the constipation treated by the compositions and methods described herein is caused by cetirizine. In some embodiments, the constipation treated by the compositions and methods described herein is caused by fexofenadine.

In some embodiments, a method for treating constipation in an individual comprises providing the individual with a composition comprising one or more food ingredients and one or more laxatives as described herein, and wherein the formulation of the composition is such that ingestion of the composition by the individual causes a bowel movement in the individual.

### Manufacture

Non-limiting examples of processes for assembling a food item as described herein include mixing, cooking, and baking one or more food ingredients and/or one or more laxatives.

When the one or more food ingredients and/or the one or more laxatives are heated in order to manufacture the food item, the heating is done at a temperature wherein the food ingredients tend to coalesce together while the laxatives remain unchanged. In some embodiments, neither the one or more food ingredients coalesce nor are the one or more laxatives changed. In some embodiments, the one or more food ingredients and the one or more laxatives all coalesce together when heated. Typically, heat is not applied at or above a temperature at which a therapeutic property of one or more laxatives of the compound are affected.

The effect of heating is determined by both the temperature and duration over which heat is applied. In some embodiments, a compound for treating constipation comprises a mixture of one or more laxatives and one or more food ingredients which are heated to a temperature at or above 350 degrees Fahrenheit. In some embodiments, a compound for treating constipation comprises a mixture of one or more laxatives and one or more food ingredients which are heated to a temperature at or above 300 degrees Fahrenheit. In some embodiments, a compound for treating constipation comprises a mixture of one or more laxatives and one or more food ingredients which are heated to a temperature at or above 250 degrees Fahrenheit. In some embodiments, a compound for treating constipation comprises a mixture of one or more laxatives and one or more food ingredients which are heated to a temperature at or above 200 degrees Fahrenheit. In some embodiments, a compound for treating constipation comprises a mixture of one or more laxatives and one or more food ingredients which are heated to a temperature at or above 150 degrees Fahrenheit. In some embodiments, a compound for treating constipation comprises a mixture of one or more laxatives and one or more food ingredients which are heated to a temperature at or above 100 degrees Fahrenheit. In some embodiments, a compound for treating constipation comprises a mixture of one or more laxatives and one or more food ingredients which are heated to a temperature at or above 95 degrees Fahrenheit. In some embodiments, a compound for treating constipation comprises a mixture of one or more laxatives and one or more food ingredients which are heated to a temperature at or above 90 degrees Fahrenheit. In some embodiments, a compound for treating constipation comprises a mixture of one or more laxatives and one or more food ingredients which are heated to a temperature at or above 85 degrees Fahrenheit. In some embodiments, a compound for treating constipation comprises a mixture of one or more laxatives and one or more food ingredients which are heated to a temperature at or above 80 degrees Fahrenheit. In some embodiments, a compound for treating constipation comprises a mixture of one or more laxatives and one or more food ingredients which are heated to a temperature at or above 75 degrees Fahrenheit. In some embodiments, the duration of heating is 1 hour or greater. In some embodiments, the duration of heating is 45 minutes or greater. In some embodiments, the duration of heating is 30 minutes or greater. In some embodiments, the duration of heating is 20 minutes or greater. In some embodiments, the duration of heating is 15 minutes or greater. In some embodiments, the duration of heating is 10 minutes or greater. In some embodiments, the duration of heating is 5 minutes or greater. In some embodiments, the duration of heating is 1 minute or greater.

In some embodiments, a compound for treating constipation further comprises a binder, wherein a binder is an element used to bind one or more elements of the compound together. In some embodiments, the binder is edible. In some embodiments, the binder comprises cocoa butter. In some embodiments, the binder comprises coconut oil.

In some embodiments, a binder is heated and the elements of the compound are added to the heated binder. In some embodiments, one or more elements of the compound are added to the binder at the hottest temperature to which it is heated. In some embodiments, one or more elements of the compound are added to the heated binder after it has cooled. In some embodiments, the binder is a liquid when heated so that when mixed with the elements of the compound, the elements coalesce or are “bound” together by the binder. In an exemplary embodiment, a binder in the compound is in a solid state at room temperature and a liquid state at a relatively small increase in temperature above room temperature. This exemplary binder is used in binding the elements of the compound when a solid compound is used such as, for example, a bar. In the liquid state the binder can coalesce the elements, and, as stated, the exemplary binder is in a liquid state at temperatures slightly above room temperature, thus the other elements of the compound are not heated due to the binder to a large extent which is especially important with respect to the laxatives that are damaged at high temperatures. In some embodiments, a binder is heated to a temperature at or above 95 degrees Fahrenheit and then one or more elements of the compound for treating constipation are mixed together with it. In some embodiments, a binder is heated to a temperature at or above 90 degrees Fahrenheit and then one or more elements of the compound for treating constipation are mixed together with it. In some embodiments, a binder is heated to a temperature at or above 85 degrees Fahrenheit and then one or more elements of the compound for treating constipation are mixed together with it. In some embodiments, a binder is heated to a temperature at or above 80 degrees Fahrenheit and then one or more elements of the compound for treating constipation are mixed together with it. In some embodiments, a binder is heated to a temperature at or above 75 degrees Fahrenheit and then one or more elements of the compound for treating constipation are mixed together with it. In some embodiments, a binder is heated to a temperature at or above 70 degrees Fahrenheit and then one or more elements of the compound for treating constipation are mixed together with it. In some embodiments, after being heated to an initial temperature, the binder is allowed to cool 30 degrees from the initial temperature before one or more elements of the compound for treating constipation are added. In some embodiments, after being heated to an initial temperature, the binder is allowed to cool 25 degrees from the initial temperature before one or more elements of the compound for treating constipation are added. In some embodiments, after being heated to an initial temperature, the binder is allowed to cool 20 degrees from the initial temperature before one or more elements of the compound for treating constipation are added. In some embodiments, after being heated to an initial temperature, the binder is allowed to cool 15 degrees from the initial temperature before one or more elements of the compound for treating constipation are added. In some embodiments, after being heated to an initial temperature, the binder is allowed to cool 10 degrees from the initial temperature before one or more elements of the compound for treating constipation are added. In some embodiments, after being heated to an initial temperature, the binder is allowed to cool 5 degrees from the initial temperature before one or more elements of the compound for treating constipation are added. It is understood that numerous other edible binders are suited, non-limiting examples of which include lard, vegetable shortening, palm oil, butter, or margarine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating constipation in an individual comprising:
providing the individual with a food item comprising one or more food ingredients and one or more laxatives, wherein ingestion of the food item by the individual produces a bowel movement in the individual and thus relieves constipation in the individual:
wherein the food item comprises a shelf-stable packaged solid food item.

2. The method of claim 1, wherein the one or more laxatives comprises any of dibasic sodium phosphate, magnesium citrate, magnesium hydroxide (milk of magnesia), magnesium sulfate (Epsom salt), monobasic sodium phosphate, sodium biphosphate, lactulose, polyethylene glycol (PEG), vitamin C, dioctyl sulfosuccinate (Docusate), bisacodyl (Dulcolax), castor oil, and sorbitol.

3. The method of claim 2, wherein the PEG comprises PEG 3350 in a quantity of 17 grams or less.

4. The method of claim 1, wherein the one or more food ingredients are mixed together with the one or more laxatives.

5. The method of claim 1, wherein the one or more laxatives comprises a coating of the food item.

6. The method of claim 1, wherein the bowel movement in the individual is produced within 12 hours of ingestion of the one or more laxatives.

7. The method of claim 1, wherein the bowel movement in the individual is produced within 24 hours.

8. The method of claim 1, wherein the food item comprises a ready to eat food bar.

9. The method of claim 1, wherein the food item comprises an edible powder either with or without flavoring and configured to be mixed with a liquid.

10. The method of claim 1, wherein the individual uses a medication, a side effect of which is constipation.

11. The method of claim 10, wherein the medication comprises an opioid.

12. The method of claim 11, wherein the opioid comprises one or more of morphine, codeine, oxycodone, and dilaudid.

13. The method of claim 10, wherein the medication comprises an antidepressant

14. The method of claim 13, wherein the antidepressant comprises one or more of amitriptyline and imipramine.

15. The method of claim 10, wherein the medication comprises an anticonvulsant.

16. The method of claim 15, wherein the anticonvulsant comprises one or more of phenytoin and carbamazepine.

17. The method of claim 10, wherein the medication comprises an iron supplement.

18. The method of claim 10, wherein the medication comprises a calcium channel blocker.

19. The method of claim 18, wherein the calcium channel blocker comprises one or more diltiazem and nifedipine.

20. The method of claim 1, wherein the constipation has lasted for a duration of 24 hours or more.

21. The method of claim 1, wherein the food item comprises a meal.

22. The method of claim 1, wherein the one or more laxatives are 50% or greater by weight of a total weight of the food item.

23. A composition comprising:
one or more laxatives;
one or more food ingredients:
wherein the laxative is combined with the one or more food ingredients, and wherein the one or more food ingredients are either of a quantity or of a type such that when the composition is ingested by an individual, the one or more food ingredients will be digested to an extent that the one or more laxatives will be released within a colon of the individual.

24. The composition of claim 23, wherein the one or more laxatives comprises polyethylene glycol (PEG) 3350.

25. The composition of claim 23, wherein the PEG 3350 comprises a quantity of 17 grams or less.

26. The composition of claim 23, wherein the one or more food ingredients are mixed together with the one or more laxatives.

27. The composition of claim 23, wherein the one or more food ingredients comprise a coating of the composition.

28. The composition of claim 23, wherein a bowel movement in the individual is produced within 12 hours of ingestion of the composition.

29. The composition of claim 23, wherein the composition comprises a ready to eat food bar.

30. The composition of claim 23, wherein the composition comprises a ready to drink shake.

31. The composition of claim 23, wherein the composition comprises an edible powder configured to be mixed with a liquid.

32. The composition of claim 23, wherein the composition comprises a meal.

33. The composition of claim 23, wherein the one or more laxatives are 50% or greater by weight of a total weight of the composition.
